(19) United States
(12) Patent Application Publication
(10) Pub. No.: US 2009/0247805 A1
(43) Pub. Date: Oct. 1, 2009

(54) Method For Producing A High-Octane Gasoline From A C5/C6 Fraction By Means Of A Membrane Separation Unit

(76) Inventors: Laurent Bournay, Chaussan (FR); Elsa Jolimaitre, Lyon (FR); Arnaud Baudot, Lyon (FR); Jean-Francois Joly, Lyon (FR); Paul Broutin, Chaponost (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/579,206

(22) PCT Filed: Nov. 9, 2004
(86) PCT No.: PCT/FR04/02885

§ 371 Date: Mar. 12, 2007

(30) Foreign Application Priority Data
Nov. 14, 2003 (FR) 0313400

(51) Int. Cl. C07C 5/13

(52) U.S. Cl. 585/738

(57) ABSTRACT

The invention relates to a method for isomerising typically paraffinic hydrocarbon fractions having 5-7 carbon atoms consisting in using a membrane separation unit which is supplied by an overhead flux from a deisohexaniser which makes it possible to maximise the isopentane quantity in isomerate. Said invention makes it possible to definitely improve the isomerate RON and MON indices by the inventive method.

### FIELD OF THE INVENTION

This invention describes an improved process for the production of gasoline bases with high octane numbers from hydrocarbon feedstock that has essentially 4 to 8 carbon atoms and that typically contains a majority of paraffins, whereby said process combines an isomerization reactor, and a distillation separation followed by a membrane separation. The term “in a majority” or “for the most part” means, according to the invention, that the percentage by weight is at least 50% and preferably at least 60%, whereas the expression “significant amount” means at least 20% by weight and preferably at least 30% by weight, and the expression “essentially” means at least 80% by weight, and preferably at least 90% by weight. A Cn fraction means, according to the invention, a fraction that essentially comprises hydrocarbons with n carbon atoms. A Cn+ fraction means, according to the invention, a fraction that comprises essentially hydrocarbons with at least n carbon atoms.

The invention falls within the context of the production of gasolines with high octane numbers.

From this standpoint, and taking into account limitations of aromatic compounds imposed by the new regulation (in Europe, currently 42% by volume of aromatic compounds), it is necessary that the hydrocarbons that constitute the gasoline contain branched paraffins in the largest contents possible. The octane numbers of the paraffins greatly depend on the type of isomer as the values of the research octane number (RON) and the engine octane number (MON) of various hydrocarbon compounds that are provided in the table below indicate:

| | | | Methyl | Methyl | | | | |
|---|---|---|---|---|---|---|---|---|
| | Normal | Normal | Heptane | Hexane | Dimethyl | Dimethyl | Trimethyl | Trimethyl |
| | Octane | Heptane | (Mono | (Mono | Hexane | Pentane | Butane | Pentane |
| Paraffins | (nC8) | (nC7) | C8) | C7) | (di C8) | (di C7) | (tri C7) | (tri C8) |
| RON | <0 | 0 | 21-27 | 42-52 | 55-76 | 80-93 | 112 | 100-109 |
| MON | <0 | 0 | 23-39 | 23-39 | 56-82 | 84-95 | 101 | 96-100 |

The abbreviations “mono,” “di” and “tri” respectively designate the paraffins with 1 branch (1 tertiary carbon), 2 branches or di-branched paraffins (comprising either 2 atoms of tertiary carbon or one atom of quaternary carbon), and the paraffins with 3 branches or tri-branched paraffins. In the text below, multibranched paraffins are defined as paraffins that have at least two degrees of branches (for example di-branched C6=paraffins with 6 carbon atoms in all, with two branches).

The octane number of the C5-C6 fraction of the gasoline obtained from the distillation of crude oil is generally between 60 and 75, i.e., for the most part lower than the standards in force.

The process that is generally used to increase the octane number of the C5-C6 fraction is the isomerization that makes it possible to transform the normal paraffins with a low octane number into branched paraffins with a high octane number.

The isomerization reaction being limited by a thermodynamic equilibrium, there is always a certain proportion of normal paraffins at the outlet of the isomerization reactor that limits the octane number of the isomerate that is produced (effluent of the isomerization unit) to values of generally between 80 and 90.

### PRIOR ART

The solution that is generally used to increase the octane number of the isomerate consists in recycling the compounds with low octane numbers (normal paraffins, and preferably also mono-branched paraffins with 6 carbon atoms) that are not converted at the top of the isomerization reactor after having separated them from the isomerate. Several separation techniques are used and known to one skilled in the art. Thus, it is possible to use differential adsorption properties of normal paraffins and iso paraffins on a suitable molecular sieve.

Thus, the U.S. Pat. No. 4,210,771 and EP-0 524 047 describe processes that combine an isomerization and a separation by gaseous phase adsorption making it possible to recycle all the normal paraffins at the top of the isomerization reactor.

Patents, such as the U.S. Pat. No. 5,602,291, that propose recycling the normal paraffins but also the mono-branched paraffins with 6 carbon atoms at the same time, which makes it possible to obtain a further improved octane number of the isomerate, are also found.

All of these processes are based on the use of adsorption processes that are well known to one skilled in the art, such as the PSA process (“Pressure Swing Adsorption” that it is possible to translate by adsorption process with pressure variation) or the so-called simulated counter-current process (CSS) or simulated moving bed.

Another possibility for carrying out the separation of normal paraffins at the outlet of the isomerization reactor is to use a distillation column that is called a deisohexanizer (DIH) that makes it possible to recycle specifically the normal hexane and the mono-branched hexane of C6 in the isomerization reactor. It is also possible to use several successive distillation columns.

Patent EP-1 205 460 describes a process for separation of a flow that contains at least 2 and 3 methylpentane, 2,2 and 2,3 dimethybutane, and isopentane, methylcyclopentane, cyclohexane and hydrocarbons of C7+ into three effluents that use a (separative) partition column; whereby the first flow contains 2 and 3 methylpentane in draw-off from the second fractionation zone of the partition column, and the second flow contains the 2,2 dimethylbutane and at least a portion of the 2,3 dimethylbutane as well as the isopentane that is extracted at one end of the column, and the third flow contains the methylcyclopentane, the cyclohexane and the C7+ at the bottom of the partition column.

Nevertheless, this process, less expensive than the adsorption processes, exhibits the drawback of not recycling the normal pentane that is found at the top of the deisohexanizer mixed with the isopentane, which significantly reduces the octane number of the isomerate.

U.S. Pat. No. 5,146,037 notes the use of a PSA technology for extracting the normal pentane from the distillate of a deisohexanizer. The PSA-type processes require, however, relatively increased investments by the complexity of their operation and significant maintenance fees.

Actually, these processes operate according to an alternation, at high frequency, of adsorption stages (of a duration of generally between one minute and one hour according to the processes, and the adsorbent amount used), and regeneration stages, at lower pressure.

In addition, it is difficult with the PSA-type processes to adapt to a variation in the flow rate or in the composition of the feedstock or else to the aging of the sieve, so as to keep performances identical, for example in terms of RON.

### PRESENTATION OF THE INVENTION

FIG. 1 shows an example of an overall diagram of the process according to the invention with its main elements: the isomerization unit, the stabilization column, the deisohexanizer and the membrane separation unit.

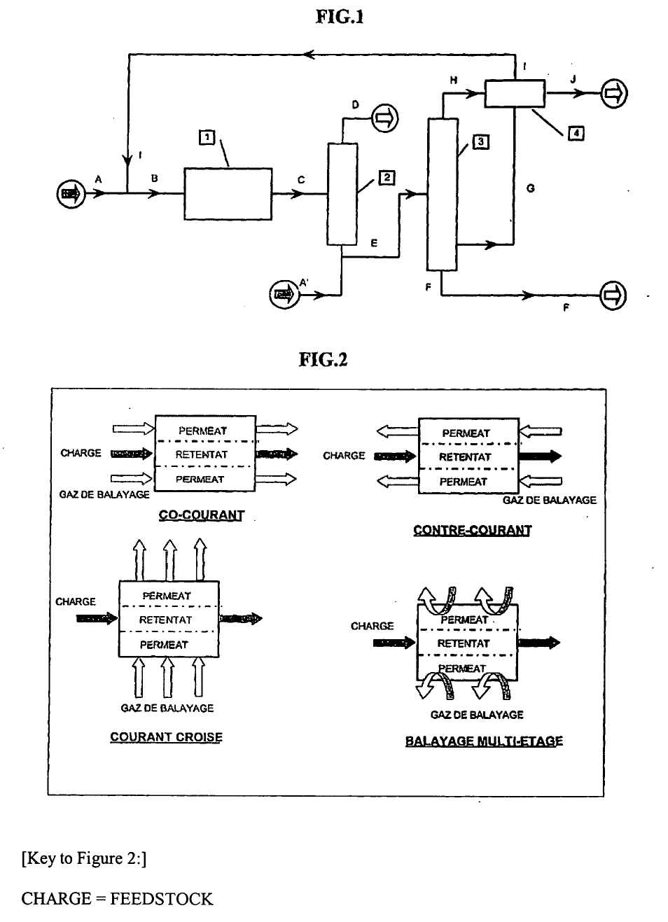

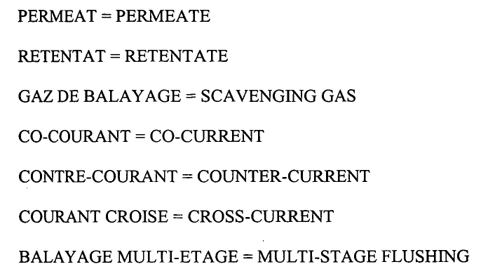

FIG. 2 provides a diagrammatic representation of the various implementations of the scavenging gas at the membrane separation unit.

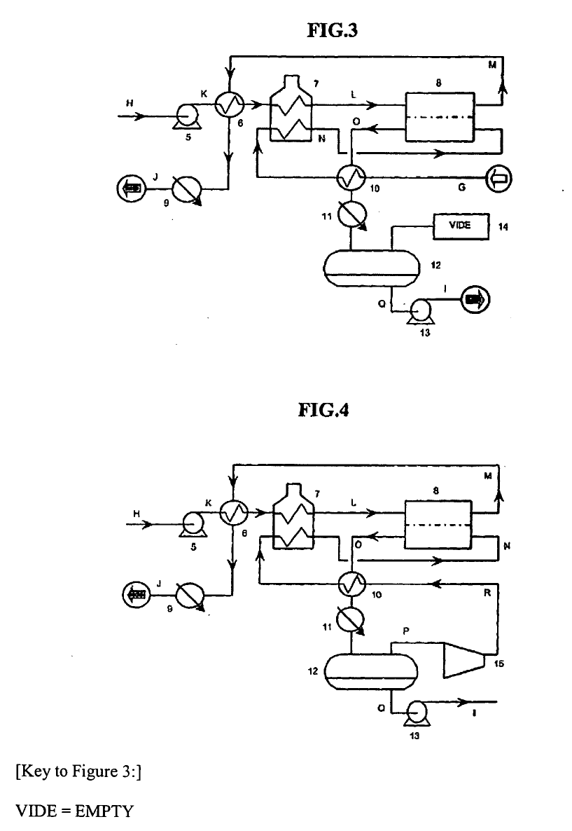

FIG. 3 corresponds to a diagram of the membrane separation unit in a variant in which the scavenging gas consists of hydrocarbons that can be recycled in the isomerization unit.

FIG. 4 corresponds to a diagram of the membrane separation unit in a variant in which the scavenging gas consists of incondensable products.

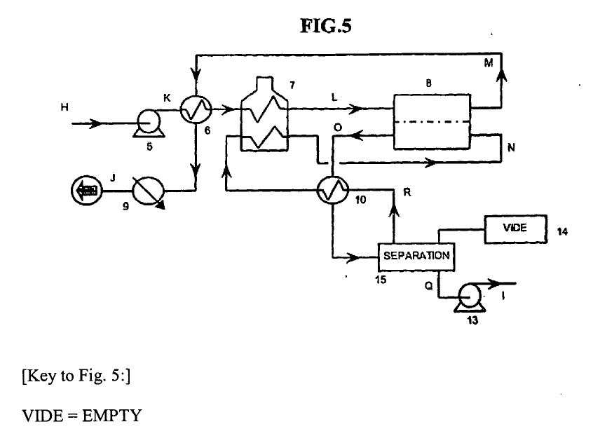

FIG. 5 corresponds to a diagram of the membrane separation unit in a variant in which the scavenging gas consists of hydrocarbons that cannot be recycled in the isomerization unit.

The invention relates to a process for the production of gasoline with a high octane number (for example the one that is shown in FIG. 1) from a hydrocarbon feedstock that for the most part has 5 to 7 carbon atoms, containing a majority of normal paraffins, iso-paraffins, and naphthenic compounds, and, accordingly, a minority of aromatic compounds, in which at least a portion of the feedstock and/or the feedstock after separation of at least a portion of branched paraffins is introduced into an isomerization unit (1), and an effluent (C) that is enriched with multi-branched paraffins is recovered, and the effluent (C) is sent into a stabilization column (2) from where light gases (D) that comprise hydrocarbons that have fewer than 5 carbon atoms are taken out at the top, and a flow (E) that is sent into a distillation column that is called a deisohexanizer (3) is taken out at the bottom, from which at least two flows are extracted:
a) At the top: a flow (H) that contains for the most part or essentially a mixture of normal pentane, isopentane and di-branched C6 paraffins,
b) In lateral draw-off or at the bottom: a flow (G) that comprises for the most part or essentially normal hexane and mono-branched C6 paraffins, which is, at least in part, recycled to the isomerization unit (1) and/or sent to a zone for storing and mixing petrochemical naphtha,
c) Optionally, at the bottom of the column, a flow (F) that contains a majority of C7 branched paraffins, cyclohexane and naphthenes, and in which the top flow (H) is directed at least in part toward a separation unit (4) by at least one selective membrane relative to the normal pentane/isopentane separation, from which is extracted a retentate (J) that is low in normal pentane and that contains for the most part or essentially isopentane and di-branched C6 paraffins, which is directed toward a zone for storing and mixing gasoline, and a permeate (I) that comprises a significant amount or a majority of normal pentane, which, at least in part, is recycled to the isomerization unit (1) and/or is sent to a zone for storing and mixing petrochemical naphtha. The term “zone for storing and mixing” is better known in its English translation: “pool,” and refers to a zone for storing and mixing with other components, to form a commercial product (for example gasoline for the gasoline pool). The term “petrochemical naphtha” refers to a steam-cracking feedstock.

The hydrocarbon feedstock is sometimes introduced at least in part at the stabilization column (2) and/or at the deisohexanizer (3), so as to reduce the supply of the isomerization unit. Advantageously, at the membrane separation unit (4), a scavenging gas of the permeate that comprises a hydrocarbon and/or a hydrocarbon mixture is used, whereby this gas can also contain hydrogen, and a mixture that comprises this hydrocarbon or these hydrocarbons with the permeate is recovered, at the outlet of the membrane separation unit, that is recycled at least in part to the isomerization unit and/or that is sent to the zone for the storing and mixing (pool) of petrochemical naphtha.

Paraffins with few or no branches are typically used as a scavenging gas to promote the permeation of normal pentane through the membrane, as will be explained below.

According to one of the preferred variants of the process according to the invention, the scavenging gas of the permeate that is used at the membrane separation unit comprises at least a portion of the flow G, which typically comprises normal hexane and mono-branched hexanes (typically as well as a small amount of 2.3 dimethylbutane, difficult to separate). An intensive permeation/distillation integration thus is achieved by using paraffins with few or no branches that are recovered by distillation to assist in the permeation of the normal pentane through the membrane. According to another preferred variant, carrying out another advantageous integration mode, the scavenging gas of the permeate that is used at the membrane separation unit comprises a hydrogen-rich gas that is used in series for the flushing of the membrane and then the dilution of the isomerization feedstock. The same hydrogen loop, typically with a single compressor, then has a dual functional role to assist in the flushing of the membrane and the dilution with hydrogen at the isomerization (function of assistance to isomerization and protection of the catalyst). The recycling loop then typically includes the membrane separation unit and the isomerization unit. It is also possible, alternately, to use a portion or all of the hydrogen that is used for the isomerization as a scavenging gas of the direct passage membrane separation, without recycling.

Advantageously, it is possible to use an operating pressure of the permeate, at the membrane separation, that is slightly greater (for example from 0.001 to 0.2 MPa) than the input pressure of the isomerization to supply the isomerization directly by natural flow (without depressurization or repressurization), preferably without hydrocarbon condensation.

The scavenging gas that is used at the membrane separation unit often operates in cross-current or in counter-current, which may or may not have multiple stages.

The membrane separation can be of vapor permeation type (retentate and vapor permeate) or of pervaporation type (liquid retentate, vapor permeate). It can also use a hyperbaric membrane process of the hyperfiltration, nanofiltration or reverse osmosis type.

It is possible, for example, to use an MFI- and/or ZSM-5-type zeolite-based membrane, whereby said zeolites are native or have been exchanged with ions of the group that consists of the ions: H+; Na+; K+; Cs+; Ca+; and Ba+.

It is also possible to use a membrane with an LTA-type zeolite base or a polymer membrane, or a composite that consists of polymers and at least one inorganic material.

The extracted linear paraffins, in the process according to the invention, of the membrane separation unit, i.e., essentially the normal pentane, are preferably recycled, partly or completely, to the isomerization section so as to be converted into compounds with a higher degree of branching and having a better octane number.

According to an alternative variant of the invention, these linear paraffins can be sent for mixing to a zone for storing and mixing (pool) of petrochemical naphtha used for the steam-cracking. The linear paraffins and/or mono-branched paraffins actually provide very good ethylene yields by steam-cracking that are higher by several points than those of a conventional naphtha. The invention also relates to a steam-cracking base that comprises, for the most part or essentially, normal hexane and mono-branched hexanes, or else normal pentane, normal hexane and mono-branched hexanes, whereby these compounds are produced by the process according to the invention. It is also possible to use these linear paraffins and/or mono-branched paraffins (included in the flow (G) and/or in the permeate) in part as a steam-cracking base, and in part as recycling to the isomerization.

### DETAILED DESCRIPTION OF THE INVENTION

A typical diagram of the implementation of the process according to the invention is shown in FIG. 1:

A flow (A) of feedstock, for example a C5/C6/C7 fraction, is supplemented with a recycling flow (I), comprising for the most part and generally essentially, normal pentane, normal hexane and mono-branched hexanes. It can also comprise small amounts of 2-methylpentane. The resulting flow (B) is isomerized in an isomerization unit (1), from which an effluent (C) that supplies a stabilization column (2) exits. The isomerization is conducted in the presence of a hydrogen flow, not shown. The column (2) produces a light gas (D) that essentially comprises hydrocarbons with at most 4 carbon atoms and the residual hydrogen at the top and a flow (E), after optional addition of another portion (A′) of the feedstock, at the bottom. The flow (E) supplies a partition deisohexanizer (3) for producing three flows: at the top, a flow (H) that consists primarily or essentially of (iso and normal) pentanes and the largest portion of the di-branched hexanes (2,2 and 2,3-dimetyl butane); a lateral draw-off flow (G) that consists primarily or essentially of normal hexane and mono-branched hexanes (2 and 3-methyl-pentane); finally, at the bottom, a flow (F) that consists primarily or essentially of C7 branched paraffins, cyclohexane, and naphthenes (and optionally small amounts of benzene). This flow (F) advantageously can supply the gasoline pool of the refinery because its octane number is acceptable. The top flow (H) is supplied with the separation unit (4) by selective membrane (4), using the lateral draw-off (G), after evaporation, as scavenging gas. The isomerization hydrogen can be supplied at this level. The separation unit (4) makes it possible to obtain a retentate (J) that is very low in normal pentane and consists for the most part or essentially of the isopentane and di-branched hexanes. This high octane fraction is sent to the gasoline pool. The flow (I) of permeate, which comprises the scavenging gas, is recycled to the isomerization. The essential elements for the implementation of the process according to the invention are presented in detail below:

### Isomerization Unit:

The processes for isomerization of fractions most often comprising paraffins with 5 and 6 carbon atoms and sometimes able to comprise paraffins with 4 and/or 7 or even 8 carbon atoms are well known to one skilled in the art. They generally use a catalyst that is selected from among three types of different catalysts:
the Friedel and Crafts-type catalysts, such as the catalysts that contain aluminum chloride, which are used at low temperature (about 20 to 130° C.),
the metal/substrate bifunctional catalysts based on metals of group VIII of the periodic table (Handbook of Chemistry and Physics, 45^{th} Edition, 1964-1965), deposited on alumina, typically platinum (often 0.25 to 0.4% by weight of platinum) and generally containing a halogen, for example chlorine and/or fluorine, which are used at average temperatures (about 110° C. to 160° C.) when they contain a halogen, or at high temperatures (350° C. to 550° C.) if not. The U.S. Pat. No. 2,906,798, U.S. Pat. No. 2,993,398, U.S. Pat. No. 3,791,960, U.S. Pat. No. 4,113,789, U.S. Pat. No. 4,149,993, and U.S. Pat. No. 4,804,803 describe, for example, these types of catalysts. It is also possible to cite other patents that have as an object monometallic catalysts with a platinum base deposited on a halogenated alumina, and their use in processes for isomerization of normal paraffins: the U.S. Pat. No. 3,963,643, which imposes a treatment by a Friedel and Crafts-type compound followed by a treatment with a chlorinated compound that comprises at least two chlorine atoms, whereby this treatment applies more particularly to the linear-chain hydrocarbons containing 4 to 6 carbon atoms. The U.S. Pat. No. 5,166,121 describes a catalyst that comprises the gamma-alumina shaped in the form of balls and comprising between 0.1 and 3.5% by weight of halogen on the substrate. The content of halogen, preferably of chlorine, deposited on the substrate, is therefore relatively low, and other catalysts contain 5 to 12% by weight of chlorine. The catalysts that comprise a halogen require the pretreatment of the feedstock because they are very sensitive to poisons and in particular to water. They are, moreover, relatively more difficult to use, often requiring the injection of a halogenated compound, which generates corrosion. The processes with a platinum-type catalyst on chlorinated alumina are often operated either in gaseous phase, with a hydrogen to hydrocarbon molar ratio (H2/HC) that is higher than 0.5, for example 0.8 (often with hydrogen recycling); under a pressure of about 2 MPa, or in a mixed phase, with H2/HC less than 0.1, for example 0.05, and even less (often without hydrogen recycling) and a pressure of about 3 MPa.
The bifunctional zeolitic catalysts that comprise a metal of group VIII that is deposited on a zeolite, which are used at high temperatures (from 250° C. to 350° C.). These catalysts lead to obtaining a mixture of hydrocarbons having an octane number that is improved but not as good as the one obtained by the processes using the catalysts cited above; however, they offer the advantage of being easier to use and more resistant to poisons. Their low acidity does not make it possible to use them for the isomerization of n-butane. These catalysts offer the advantage of being very easy to use, and of being resistant to poisons such as sulfur and water, which prevents a pretreatment of the feedstock. They are also frequently used. The U.S. Pat. No. 4,727,217 describes these types of catalysts.

The actual processes for isomerization of paraffins containing 5 and 6 carbon atoms often use chlorinated-alumina-type catalysts comprising platinum, which are high-activity catalysts. These processes are used without recycling (in English, “once through”) or with a partial recycling after fractionation of the unconverted normal paraffins, for example by distillation(s) or else with a total recycling after passage over molecular sieve systems in liquid phase. These processes lead to obtaining a base for fuels that often contain few or no aromatic compounds (generally less than 20% by weight, and most often less than 2% by weight), and whose research octane number (RON) is generally between 82 and 88.

The invention is not limited to a catalyst and/or to a process for isomerization of particular light paraffins, but can be used with any type of catalyst and any process. It will be possible to use in particular a process with an operating pressure of between 0.1 and 10 MPa, a temperature of between 90 and 400° C., and an H2/HC molar ratio of between 0.001 and 3, and any type of catalyst for isomerization of light paraffins, in a gaseous, mixed, or liquid phase, with or without recycling of hydrogen, in one or more stages, with any type of feedstock that comprises significant amounts (for example 30 to 95%) by weight of paraffins that have 4 to 8 carbon atoms, inclusive. The paraffins can be obtained from direct distillation fractions for petroleum, and/or cracking (cracking catalytic fluid, steam-cracking, delayed coking or coking in a fluidized bed, viscoreduction), with or without previous hydrogenation, and/or catalytic reforming, and/or Fischer-Tropsch synthesis.

### Deisohexanizer:

The deisohexanizer is often, in particular when the feedstock is a standard feedstock (typically essentially C5/C6, with a benzene content of less than 2% by weight), a standard distillation column with one inlet and two outlets, one at the top (essentially C5+[di-branched C6]) and the other at the bottom (primarily normal hexane and mono-branched C6).

It is also possible to use a distillation column with separative partition(s) from which are drawn off at least three flows: (H) at the top, (G) in a lateral draw-off, and (F) at the bottom. A detailed description of this type of partition column, by Howard Rudd, can be found in the magazine supplement “The Chemical Engineer” (L'Ingénieur chimiste), Editor: “Institution of Chemical Engineers,” Davis Building, 165-171 Railway Terrace, Rugby, Warwickshire CV21 3HQ, England, of Aug. 27, 1992. It is also possible to refer to the Patent EP-1 205 460. This technical option can be used in particular when a feedstock that comprises C7 hydrocarbons is isomerized.

### Selective Membrane Separation Unit:

The process according to the invention uses at least one isomerization zone and at least one separation section comprising several units of which at least one operates with a membrane.

The membrane separation offers numerous advantages:

The principle of the membrane separation is based on a shape and/or size selectivity of the molecules. It is possible to use, according to the invention, any type of membrane that exhibits a selectivity, typically of shape, between the linear light paraffins and the branched light paraffins (having 5 or 6 carbon atoms) and in particular any membrane that exhibits a noteworthy or significant selectivity relative to the isopentane/normal pentane separation. Membranes that exhibit a ratio of permeation speed of the normal pentane to the permeation speed of the isopentane that is higher than 3, preferably higher than 8, for example included between 8 and 1000, are typically used.

A membrane process in the case of pervaporation, vapor (in phase) permeation, hyperfiltration or reverse osmosis, or nanofiltration, can advantageously replace the distillation separation in the case of the separation of isomers whose boiling points are very close. Actually, the isomer separation by distillation requires the implementation of a significant separator power that will be reflected by a large number of theoretical plates and large amounts of energy for condensation and reboiling, while the membrane separation only entrains a very low energy consumption. By definition, reverse osmosis, also called hyperfiltration, is a transport of selective material in the liquid phase induced by a mechanical pressure difference through a membrane with an equivalent diameter of mean pores of less than 1.5 nanometers, and the nanofiltration is a transport of selective material in the liquid phase induced by a mechanical pressure difference through a membrane with an equivalent mean pore diameter of between 0.8 and 8 nanometers.

Another advantage of the membrane techniques is the modularity, because it is possible to adjust the purity of the retentate or the flow rate of the treated feedstock thanks to the membrane surface that is used, or by the number of modules used, without increasing the energy consumption and the utilities consumption.

This modularity also makes it possible to manage the replacement or the regeneration in situ of membrane modules (for example for reasons of aging of the material) without stopping the production.

It is therefore natural to consider replacing the conventional technique for separating linear paraffins (by distillation(s)) by a selective membrane separation. Such a separation makes it possible to separate simultaneously both the C5 linear paraffins (normal pentane) and those of C6 (normal hexane), whereby the known membranes have a significant normal/iso selectivity both for the C5 paraffins and the C6 paraffins. The applicant, however, found a process implementing a particular combination of separative stages: membrane distillation/separation, exhibiting, in a surprising way, important advantages relative to each of the two separative techniques considered separately:

The use of a membrane makes it possible to reduce greatly the energy consumption relative to a process that carries out a complete fractionation by distillations, including a depentanizer (normal pentane/isopentane distillation). Relative to a complete membrane fractionation, the distillation portion makes possible the elimination or generally the recycling of mono-branched C6 paraffins whose octane number is limited. The combination of the two separations according to the invention therefore makes it possible to reduce the energy consumption of a separation entirely by distillations, while preserving an excellent effectiveness in terms of the octane number of the isomerate.

According to the type of membrane selected, the feedstock at the inlet of the membrane separation stage can be in liquid, vapor, mixed liquid/vapor, or supercritical form. On the permeate side, a liquid phase, a mixed liquid/vapor phase or preferably a vapor phase is selected.

A membrane separation of vapor permeation type (vapor phase on the permeate and retentate side) is actually particularly well suited for carrying out the separation of n/iso paraffins described in this invention.

The membrane permeating device (membrane separator) is then operated in gaseous phase, whereby the absolute pressure on the retentate side is between 0.1 and 10 MPa, and preferably between 0.5 and 3 MPa.

These parameters should be coordinated to obtain a vapor phase.

The temperature on the retentate side is typically between 50 and 500° C. and preferably between 150 and 350° C. The temperature deviation between permeate and retentate should preferably be reduced because the material that constitutes the substrate of the membrane is sensitive to the temperature gradients.

The membrane permeation is a separation process that is both simple and reliable because it does not involve mobile and economical mechanical parts.

This is a continuous process, which involves lower maintenance costs than a PSA technology.

There are different arrangements and possibilities for implementing these modules so as to optimize the flow of material through the membrane and the selectivity.

It is known to one skilled in the art that to improve the flow through the membrane, it is necessary to maximize the driving force that brings about the transfer of material through the membrane that depends directly on the partial pressure difference of the chemical radicals that are between the permeate and the retentate.

From this standpoint, it is possible to reduce the pressure of the permeate below the atmospheric pressure by putting it under partial vacuum until a value often of between 0.01 and 0.09 MPa is reached.

Actually, by lowering the total pressure on the permeate side, the deviation of partial pressure of the radicals that permeate, in particular the normal pentane, is maximized.

Another way of further improving the flow through the membrane consists in using a scavenging gas that acts as a diluent of the permeate, which has the result of lowering the partial pressure on the downstream side. The ratio of the molar flow rates of feedstock to scavenging gas is typically between 0.1 and 100, and preferably between 0.3 and 10.

This scavenging gas can be injected in co-current of the retentate, or else in counter-current, or else in cross-current.

It is also possible to carry out several flushing stages.

The schematic diagrams of these different flows are presented in FIG. 2.

Based on the presence or absence of scavenging gas and its nature, the diagram of the process can vary. These variants do not change the nature of the invention because they influence only the scavenging circuit and not the structural and functional arrangement of the process according to the invention.

The primary variants that relate to the nature and the organization of the scavenging gas are as follows:
a) The scavenging gas can comprise hydrocarbons with 5, 6 and 7 carbon atoms, preferably enriched with normal paraffins that it is possible to send as a feedstock to the isomerization with the permeate (primarily n-pentane). It is then preferable that the permeate-side pressure be low, for example less than 0.3 MPa or 0.2 MPa, or even sub-atmospheric, so that these n-paraffins do not diffuse, or diffuse very little, toward the retentate, which would be contrary to the desired objective.

In a preferred variant, this scavenging gas comprises a portion or all of the lateral draw-off of the noted DIH (G) in FIG. 1, or of the bottom draw-off (G) when the column comprises only 2 outlets, whereby this draw-off (G) typically comprises a majority, or at least 80% by weight, or essentially normal hexane and mono-branched C6.

According to FIG. 3, the so-called scavenging flow (G) of hydrocarbons is evaporated and heated in the heat exchanger (10) and the furnace (7) up to, for example, the temperature of the flow (L) of the supply of the membrane separation, encompassed between 50° C. and 500° C., and preferably between 150° C. and 350° C., then the flow (N) that is thus obtained flushes the permeate-side membrane.

The flow (O) that contains the scavenging gas and the radicals that passed through the membrane is cooled and condensed essentially completely within the heat exchangers (10) and (11), and then sent into a gas-liquid separator tank (12), whose pressure is kept subatmospheric thanks to the vacuum unit (14).

The liquid phase (Q) that is extracted from the decanting tank constitutes the flow (I) that is sent via the pump (13) upstream from the isomerization zone. The top flow (H) of the deisohexanizer is pumped by the pump (5) to obtain the flow (K), heated and evaporated in the exchanger (6) and the furnace (7) to obtain a supply (L) from the membrane separator (8). The retentate with a high octane number that is obtained from (8) is cooled in the heat exchangers (6) and (9) to obtain the flow (J) that is sent to the gasoline pool.
b) The scavenging gas can also be an incondensable product, for example a mixture that comprises at least one of the following elements: hydrogen, methane, or ethane. FIG. 4 illustrates this variant:

The flow (R) of the scavenging gas is heated in the exchanger (10) and the furnace (7) up to about the temperature of the flow (L), encompassed between 50° C. and 500° C. and preferably between 150° C. and 350° C., then the flow (N) that is thus obtained flushes the permeate-side membrane.

The flow (O) that comprises this gas and the radicals that passed through the membrane is cooled and partially condensed in the exchangers (10) and (11) up to a temperature that makes possible the gas/liquid separation of the radicals with at least 5 carbon atoms that passed through the membrane and the scavenging gas whose condensation temperature is often much lower.

At the outlet of the separator tank (12), a liquid (Q) that is pumped and recycled upstream from the isomerization zone, and a gaseous flow (P) that is compressed by the compressor (15) and recycled to the permeating device (8) are recovered.

As a variant, it is possible to supply as a scavenging gas a hydrogen-rich gas, which at the outlet of the permeating device directly supplies the isomerization unit, preferably by natural flow, without hydrocarbon condensation. This scavenging gas can then be recovered at the top of the stabilization column, optionally purified by condensation and the elimination of propane and/or butane and/or other light hydrocarbons, then, after recompression, recycled to flush the membrane.

Another option consists in not recycling this flow of hydrogen and/or incondensable products by sampling the flushing flow rate necessary for the network of hydrogen or combustible gas from the refinery or an adjacent unit. After separation of the radicals with at least 5 carbon atoms that passed through the membrane, the incondensable products can then be sent to the torch or to the fuel gas network. This option offers the advantage to eliminate one compressor by the use of a scavenging circuit with recycling.
c) The scavenging gas can also be a mixture of hydrocarbons that cannot be recycled to the isomerization. These hydrocarbons can be of all types with any distributions in the chemical family and have a number of carbon atoms of typically between 1 and 18. However, it is ensured that the partial pressures of n-paraffins on the permeate side are in particular lower (for example, by at least 0.5 MPa or even 1 to 3 MPa) than the corresponding partial pressure of n-paraffins on the retentate side.

According to FIG. 5, the hydrocarbon flow (R) is evaporated and heated in the exchanger (10) and the furnace (7) up to the temperature of the flow (L), encompassed between 50° C. and 500° C., and preferably between 150° C. and 350° C., then the flow (N) that is thus obtained flushes the permeate-side membrane. The flow (0) that contains the scavenging gas and the radicals that passed through the membrane is cooled in the exchanger (10) and sent to a separation section (15).

At the outlet of the separation section (15), the flow (R) that consists of hydrocarbons used for flushing and recycled to permeating device (8) and a flow (Q) primarily consisting of radicals with 5 carbon atoms that passed through the membrane, recycled via the pump (13) upstream from the isomerization zone, are obtained.

The separation section (15) can use any, or several, techniques for separating known hydrocarbons from one skilled in the art, such as the distillation and/or the liquid-vapor separation.

Any type of membrane that makes it possible to make the separation between the linear paraffins and the branched paraffins, whether the organic membranes or polymer membranes (for example, the PDMS 1060 membrane of Sulzer Chemtech Membrane Systems, Friedrichsthaler Strasse 19, D-66540, Neunkirchen, Germany), inorganic, ceramic or mineral membranes (composed of, for example, at least in part zeolite, silica, alumina, glass or carbon), or composites consisting of polymer and at least one inorganic compound (for example, the PDMS membrane 1070 of Sulzer Chemtech Membrane Systems), can be used within the scope of this invention.

Numerous works of literature make reference to membranes based on MFI-type zeolitic films, which make it possible to separate very effectively the linear paraffins from the branched paraffins, thanks to a diffusional selectivity mechanism.

All of the membrane types with a base of MFI zeolites have an n/isoparaffin selectivity, in particular for the normal pentane-isopentane separation, whether the membranes with a silicate base based on a completely dealuminified MFI zeolite (Vroon et al. “Transport Properties of Alkanes through Ceramic Thin Zeolites MFI Membranes” (properties of transport of alkane through fine ceramic membranes of MFI zeolite), the journal “Journal of Membrane Science” (Revue sur la Science des Membranes, Editor: Elsevier Science B.V., P.O. Box 211, 1000 AE Amsterdam, The Netherlands), 113, 1996, 293-300; Van de Graaf et al: “Effect of Operating Conditions and Membrane Quality on the Separation Performances of Composite Silicalite-1 Membranes,” the journal “Industrial Engineering Chemistry Research (Recherche en Ingéniérie Chimique Industrielle, Editor: American Chemical Society, 1155 16^{th} Street, N. W. Washington D.C. 20036, USA), 37, 1998, 4071-4083) or those based on native ZSM-5 zeolites (Coronas et al: “Separations of C4 and C6 Isomers in ZSM-5 Tubular Membranes, “the journal “Industrial Engineering Chemical Research,” mentioned above, 3, 1998, 166-176) or those that have been exchanged with ions such as H+, Na+, K+, Cs+, Ca+ or Ba+ (Aoki et al.: “Gas Permeation Pròperties of Ion-Exchanged ZSM-5 Zeolite Membranes” (Propriétés de Perméation Gazeuse des membranes zéolithiques ZSM-5 échangées par échange d'ions), the journal “Microporous Mesoporous Materials” (Materiaux microporeux et mésoporeus, Editor: Elsevier Science B. V., P.O. Box 211, 1000 AE Amsterdam, The Netherlands), 39, 2000, 485-492).

The published values of n-C4/I-C4 selectivity in a mixture, obtained with this type of membrane, vary between 10 (Van de Graaf et al., 1998, mentioned above) and 50 (Keizer et al., 1998, mentioned above; Vroon et al., 1996, mentioned above), according to the operating conditions.

The selectivities of separation observed with membranes with an MFI zeolite base applied to the n-hexane/dimethylbutane separation are also higher: 200 to 400 (Coronas et al., 1998, mentioned above) and even more.

It is also possible to consider using membranes with an LTA-structural-type zeolite base, a zeolite that has a very good shape selectivity relative to normal paraffins.

If all of the above-mentioned membranes are selective for the light n/iso paraffin separations and in particular for the n-pentane/isopentane separation, the selectivity and the permeability can vary in particular from one membrane to the next. One skilled in the art can preferably, for a particular membrane, determine the selectivity of the n/iso separation, in particular that of the separation: n-pentane/isopentane, as well as the usable permeation flow, by relatively simple laboratory tests.

The invention is not limited to this description, and one skilled in the art can use in particular every obvious variant, and all technical equivalents that are known or that result directly from known elements.

Thus, the scope of the invention would not be exceeded by replacing the deisohexanizer with 3 effluents by two successive distillation columns, typically: one deisohexanizer with 2 effluents, whose top outlet comprises the pentanes and the di-branched hexane, and the bottom outlet in particular comprises the normal hexane and the mono-branched hexanes, followed by a second column for fractionation of this bottom outlet, into a top current (identical and/or playing the same role as the lateral draw-off of the deisohexanizer with 3 effluents), specifically comprising the normal hexane and the mono-branched hexanes, and a bottom current that essentially comprises heavier hydrocarbons.

Likewise, the scope of the invention will not be exceeded by replacing the deisohexanizer with 3 effluents by two successive distillation columns, typically a hexanizer denormal with 2 effluents, whose bottom outlet typically comprises heavier products than the normal hexane, followed by a second fractionation column of the top current to separate specifically a new bottom current that essentially comprises the normal hexane and the mono-branched hexanes (current that is identical and/or that plays the same role as the lateral draw-off of the deisohexanizer with 3 effluents).

### EXAMPLES

### Example 1

### According to the Invention

Example 1 illustrates the invention in one of the preferred variants, in which the scavenging gas, used at the membrane, consists of the lateral draw-off of the deisohexanizer.

The material balance is obtained by computer simulation and uses the PRO II simulation program of the SIMSCI-ESSCOR Company, 26561 Rancho Parkway South, Lake Forest, Calif. 92630, USA. The composition of the different flows is provided in Table 1, the overall arrangement of the process is that of FIG. 1, and the detailed arrangement of the implementation of the membrane permeating device is that shown in FIG. 3.

The membrane that is used in the permeating device (8) is composed of a selective layer with an MFI-type zeolite base that is supported on an alumina tube (commercial reference T1 70 of the EXEKIA Company, P.O. Box 1, F-65460 Bazet, France) with a surface area of 5000 m2.

The first portion of the text of the example follows by means of FIG. 1.

The feedstock (A) with a flow rate of 62181 kg/h of hydrocarbons supplemented by 372 kg/h of hydrogen is mixed with a recycling flow (I) with a flow rate of 68761 kg/h. The resulting flow is introduced into the conventional isomerization section (1) with two reactors containing a platinum-type catalyst on chlorinated alumina, of reference IS 612 A, marketed by the AXENS Company, Rueil-Malmaison, France, where it is isomerized under 3 MPa and at 150° C.

After stabilization, the effluent (E) of the isomerization section supplies the deisohexanizer (3) with a flow rate of 128576 kg/h.

The deisohexanizer has a separation effectiveness of 60 theoretical stages and operates with a molar ratio of reflux flow to feedstock of 4.3.

The feedstock is introduced in plate 20 of the deisohexanizer.

The lateral draw-off (G) is sampled in plate 42 with a flow rate of 46998 kg/h.

This lateral draw-off (G) is used as a scavenging gas on the permeate side of the membrane to improve the flow of radicals that permeate through the membrane, as FIG. 3 illustrates.

Flow (F) of the column bottom, with a flow rate of 6579 kg/h and that contains for the most part naphthenes, is sent to the zone for the storing and mixing (pool) of gasoline. The top liquid distillate (H) with a flow rate of 75000 kg/h enters the membrane separation zone at the temperature of 37° C. and at the absolute pressure of 0.28 MPa.

The rest of the text of this example follows in FIG. 3.

This flow (H) is picked up by the pump (5) that raises its pressure to 1.3 MPa, then it is heated in the effluent-feedstock exchanger (6), evaporated and heated in the furnace (7) up to the temperature of 300° C.

The vapor flow (L) that is thus obtained supplies the membrane permeating device (8).

The retentate (M) with a flow rate of 53236 kg/h, low in normal pentane, passes into the effluent-feedstock exchanger (6) and is cooled in the cooler (9) before being sent into the gasoline pool.

The flow (G) of liquid that is drawn off at plate 42 of the deisohexanizer at the pressure of 0.36 MPa and at the temperature of 114° C. is heated in the effluent-feedstock exchanger (10) then evaporated and heated in the furnace (7) up to the temperature of 300° C.

The vapor flow (N) that results preferably should have essentially the same temperature as the flow (L) because the material of the membrane is sensitive to thermal deviations.

This flow (N) is introduced on the permeate side of the membrane with counter-current of the flow (L) in a preferred version of the invention.

The effluent (O) with a flow rate of 68761 kg/h, enriched with normal pentane, is cooled in the effluent-feedstock exchanger (10) and condensed essentially totally in the condenser (11).

The evacuation system (14) is connected to the tank (12) and maintains a pressure of 0.09 MPa.

The evacuation system (14) can have one or more stages and can use any of the techniques that are known to one skilled in the art, for example a vapor ejector, a liquid ring pump or a vacuum pump.

The liquid (l) that contains the radicals that are contained in the flow (G), including the normal hexane and the paraffins with 6 mono-branched carbon atoms (the 2 and 3-methyl pentanes) as well as the radicals that passed through the membrane, including the normal pentane, is collected at the bottom of the tank (12), picked up by the pump (13) and sent upstream from the isomerization zone (1).

Table 1 below provides the detailed compositions of the flows A; I; E; G; F; H; M

TABLE 1
Composition in % By Mass of the Flows

| | A | I | E | G | F | H | M |
|---|---|---|---|---|---|---|---|
| Isobutane | 0.0 | 0.0 | 0.1 | 0.0 | 0.0 | 0.1 | 0.1 |
| Normal Butane | 1.9 | 0.0 | 0.3 | 0.0 | 0.0 | 0.5 | 0.7 |
| Isopentane | 14.4 | 10.0 | 26.8 | 0.0 | 0.0 | 46.0 | 51.9 |
| Normal Pentane | 30.6 | 21.3 | 12.0 | 0.0 | 0.0 | 20.6 | 1.4 |
| Cyclopentane | 1.5 | 0.3 | 0.8 | 0.0 | 0.0 | 1.4 | 1.6 |
| 2,2-Dimethylbutane | 0.4 | 0.0 | 10.7 | 0.0 | 0.0 | 18.3 | 25.8 |
| 2,3-Dimethylbutane | 1.3 | 3.1 | 4.9 | 4.5 | 0.3 | 5.6 | 7.9 |
| 2-Methylpentane | 9.9 | 18.7 | 14.2 | 27.4 | 2.1 | 7.0 | 9.9 |
| 3-Methylpentane | 6.6 | 14.5 | 8.2 | 21.3 | 3.0 | 0.5 | 0.7 |
| Normal Hexane | 21.0 | 10.9 | 6.1 | 15.9 | 5.7 | 0.0 | 0.0 |
| Methylcyclopentane | 5.2 | 9.7 | 5.8 | 14.3 | 11.0 | 0.0 | 0.0 |
| Benzene | 2.4 | 0.0 | 0.0 | 0.0 | 0.0 | 0.0 | 0.0 |
| Cyclohexane | 3.3 | 10.8 | 7.5 | 15.8 | 33.1 | 0.0 | 0.0 |
| C7+ | 1.5 | 0.7 | 2.6 | 0.8 | 44.8 | 0.0 | 0.0 |

### Example 2

### According to the Prior Art, and Comparison

Table 2 below compares the performance levels of the isomerization process according to the prior art (without membrane separation) and according to the invention, all things being equal furthermore, as much in terms of the amount of catalyst and operating conditions of the isomerization reactors as in terms of characteristics of the stabilization column and the deisohexanizer.

The installation of the membrane permeating device according to the invention is accompanied by a gain of more than 4 points on the RON and the MON, for a comparable gasoline yield.

TABLE 2

| | | With a Permeating |
|---|---|---|
| | | Device |
| | Without a Permeating | (According to the |
| Flow Rate Toward Gasoline | Device (Prior Art) | Invention) |
| Pool in kg/h | 59904 | 59815 |
| RON | 86.7 | 91.0 |
| MON | 82.8 | 87.0 |
| Density in kg/m³ | 654.3 | 653.7 |

1. Process for the production of gasoline with a high octane number from a hydrocarbon feedstock that for the most part has 5 to 7 carbon atoms, comprising a majority of normal paraffins, iso-paraffins, and naphthenic compounds, and a minority of aromatic compounds, in which at least a portion of the feedstock and/or the feedstock after separation of at least a portion of branched paraffins is introduced into an isomerization unit (1), and an effluent (C) that is enriched with multi-branched paraffins is recovered, and the effluent (C) is sent into a stabilization column (2) from where light gases (D) that comprise hydrocarbons that have less than 5 carbon atoms are taken out at the top, and a flow (E) that is sent into a distillation column that is called a deisohexanizer (E) is taken out at the bottom, from which at least two flows are extracted:
a) At the top: a flow (H) that contains for the most part a mixture of normal pentane, isopentane and di-branched C6 paraffins,
b) In lateral draw-off or at the bottom: a flow (G) that comprises a majority of normal hexane and mono-branched C6 paraffins, which is, at least in part, recycled to the isomerization unit (1) and/or sent to a zone for storing and mixing petrochemical naphtha,
c) Optionally, at the bottom of the column, a flow (F) that contains a majority of C7 branched paraffins, cyclohexane and naphthenes,
then the top flow (H) is directed toward a separation unit (4) by a selective membrane relative to the normal pentane/isopentane separation, with flushing of the permeate by a gas that comprises at least one hydrocarbon and that comprises in particular:
Either at least one portion of the flow G and hydrogen,
Or an incondensable gas that comprises hydrogen or methane or ethane,
Or a gas that is rich in hydrogen that directly supplies the isomerization unit at the outlet;
a mixture of this hydrocarbon with the permeate is recovered, at the outlet of the membrane separation unit, that is recycled at least in part to the isomerization unit and/or that is sent to the zone for storing and mixing petrochemical naphtha, and a retentate (J) that is low in normal pentane, containing in a majority the isopentane and di-branched C6 paraffins, that is directed toward a zone for storing and mixing gasoline, is extracted from the separation unit (4).

2. Process according to claim 1, in which the hydrocarbon feedstock is introduced at least in part at the stabilization column (2) and/or at the deisohexanizer (3).

3. Process according to claim 1, in which the membrane separation is of the vapor permeation or pervaporation type.

4. Process according to claim 1, in which the membrane separation is a hyperbaric membrane process of the hyperfiltration or reverse osmosis or nanofiltration type.

5. Process according to claim 1, in which the membrane separation unit uses an MFI- or ZSM-5-type zeolite-based membrane, native or having been exchanged with ions of the group that consists of: H+; Na+; K+; Cs+; Ca+; and Ba+.

6. Process according to claim 1, in which the membrane separation unit uses a membrane based on LTA-type zeolites.

7. Process according to claim 1, in which the membrane separation unit uses a polymer membrane or composite constituted by polymers and at least one inorganic material.

8. Process according to claim 1, in which the deisohexanizer is a partition column from which are drawn off at least three flows: (H) at the top, (G) in lateral draw-off, and (F) at the bottom.
